(19) United States
(12) Patent Application Publication
(10) Pub. No.: US 2007/0255293 A1
(43) Pub. Date: Nov. 1, 2007

(54) Device for Collecting Small Diameter Hair Grafts

(76) Inventor: Alain Corre, Paris (FR)

Correspondence Address: ARENT FOX PLLC, 1050 CONNECTICUT AVENUE, N.W., SUITE 400, WASHINGTON, DC, 20036, US

(21) Appl. No.: 11/629,320

(22) PCT Filed: Jun. 7, 2005
(86) PCT No.: PCT/FR05/01390

§ 371 Date: Jun. 14, 2007

(30) Foreign Application Priority Data
Jun. 14, 2004 (FR) 0451169

(51) Int. Cl. A61B 17/32

(52) U.S. Cl. 606/133

(57) ABSTRACT

The invention relates to a device for collecting small diameter hair grafts comprising a portable cutting tool (1) which is consists of a tool-holder body (2), a cylindrical tool provided with a hollow axle (4) for inserting hollow needle (5, 17, 25) whose diameter ranges from 0.7 to 2 mm, a motor (3) for rotatably driving said needle (5, 17, 25) with respect to said body (2). The inventive device also comprises a graft storage container (9, 18, 22) which is connected to the body (2) and is provided with a graft input orifice (10, 19) connected to the vicinity of the rear end of the hollow needle (5, 17, 25) and a suction orifice (13, 20) connected to graft suction means which is arranged in such a way that the graft is enable to enter directly the container (9, 18, 22) by sucking said graft through the needle (5, 17, 25).

The invention concerns a device to harvest small hair grafts.

In particular, this invention is made to be used during the surgical treatment of baldness by hair transplant that consists of transplanting, in the same individual, the roots from part of the scalp located, for example, in the rim (always hairy) to a bald area.

To do this, we are familiar with the so-called strip method, that consists of cutting one or several strips of skin from the zone, for example the rim, and then drawing out these strips of skin so as to distinctly separate each hair, cutting fragments, called grafts, in these strips of skin and finally transplanting these grafts on the previously prepared bald area.

This method presents several disadvantages. First, before the transplant, a large number of steps have to be carried out, involving fastidous handling and requiring a long operating time. In addition, this method is painful for the individual concerned, and the zone from which the strips of skin have been removed never recovers its normal appearance since the cut leaves a 15 to 20 cm long scar.

We are also familiar with the so-calle punch technique that consists of cutting and removing cylindrical skin fragments of small diameter, also called grafts, typically about 0.7 mm to 2 mm, each fragment comprising 1 or 2 hair (micro-graft) or 3 to 5 hair (min-graft), then reimplanting these grafts one at a time in the previously prepared receiver sites. This technique has the advantage that it is not invalidating for the invidividual concerned and the small diameter of the grafts removed lets the skin heal very quickly.

To use this technique, from document FR-A-2 696 334 we are familiar with a device for transplant comprising a portable cutting instrument comprising a tool-rest body, a rotary cylindrical tool formed by a hollow needle called a punch and the means of removing the graft by suction of the aforementioned graft through the needle.

Moreover, this device comprises a portable graft implant device comprising a body, an intermediate storage tank for the graft, a hollow reimplantation needle and means to introduce the graft in the scalp through the hollow reimplantation needle, from the intermediate tank.

In this device, the intermediate storage tank for the graft is an integral part of the implant apparatus, indicating that each graft, once cut, not only passes through the cutting instrument, but also through the pipe connecting the cutting instrument and the implant device, thereby presenting several disadvantages.

First, the risk of the grafts blocking the aforementioned pipe is high since the length of the pipe is considerable. In addition, the circulation of the grafts in the pipe is likely to provoke their alteration, since the grafts consist of organic matter and, as a result, are extremely fragile. Also, the fact that the storage tank is located inside the implant device prevents the operator from correctly seeing the graft and as a result evaluating its quality before its implant on the bald zone of the individual concerned. In addition, the simultaneity of the removal and the implant requires very good co-ordination by both operators.

To overcome these disadvantages, the invention proposes a device to remove small capillary grafts in which a graft storage tank is integral with the body of the portable cutting instrument, not only avoiding the risk of blodking the pipe but also avoiding any alteration in the grafts after they are cut. In addition, the efficacy of the suction of the aforementioned grafts is increased, since the tank is placed between the end of an inner needle and the means of suction. Therefore, such an arrangement presents advantages not only in terms of quality of the implanted grafts, but also in terms of the rapidity of removal.

For this purpose, the invention proposes a device to remove small hair grafts comprising a portable cutting instrument, the aforementioned instrument comprising:
a tool-rest body;
a cylindrical tool with a hollow axis enabling the insertion of a hollow needle whose diameter ranges from 0.7 mm to 2 mm;
a motor able to drive the aforementioned needle in rotation with respect to the aforementioned body, the aforementioned device also comprising a graft storage tank integral with the aforementioned body, the aforementioned tank comprising a graft inlet connected near the rear end of the hollow needle and a suction opening connected to means of suction of the graft, since the aforementioned means of suction are arranged to allow the graft to directly reach the tank by suction of the aforementioned graft through the needle.

According to one design, the tool-rest body is a roughly cylindrical handpiece.

According to another design, the tool-rest body is a handpiece such as a contra-angle handpiece. This design presents an advantage in terms of cost since such a handpiece can be made from apparatus that is currently used, in particular in dental surgery, and as a result are commercially available. In addition, the hollow axis foreseen for the insertion of the needle and the passage of the grafts by suction may also be made independent of the aforementioned storage tank.

Other objects and advantages of the invention will appear in the following description, referring to the appended drawings, in which:

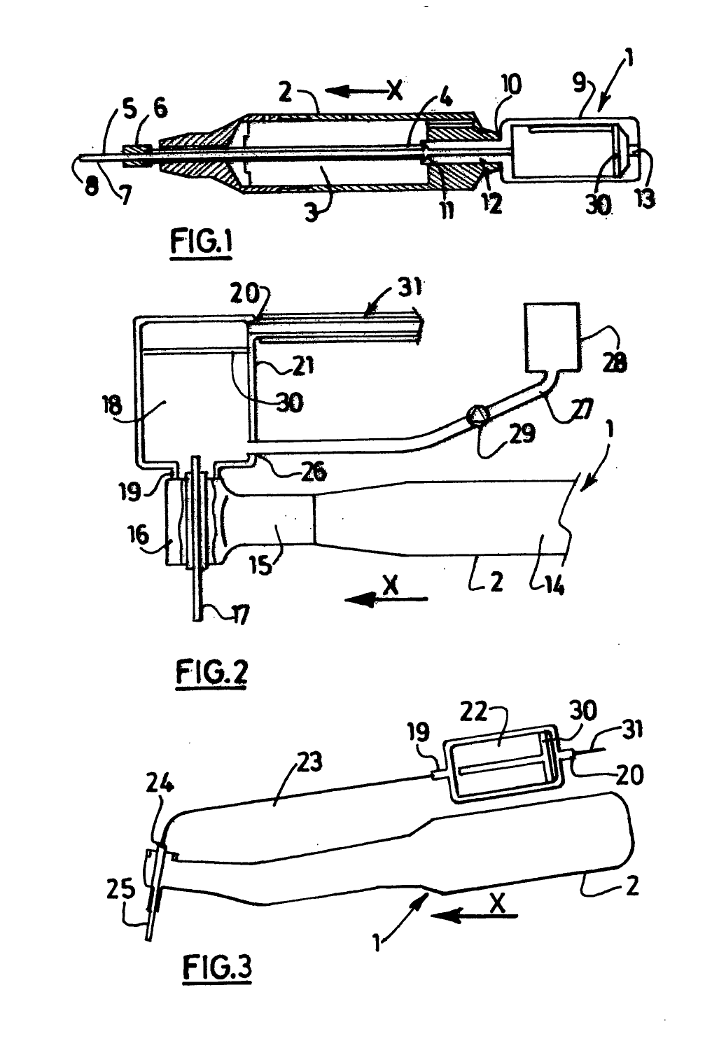

FIG. 1 is a longitudinal section of a device according to the invention, according to a first design;

FIG. 2 is a longitudinal section of a device according to the invention, according to a second design;

FIG. 3 is a longitudinal section of a device according to the invention, according to a third design.

The device according to the invention is intended for the removal of small hair grafts. By small, we mean a diameter ranging from about 0.7 mm to about 2 mm.

In the following description, “front” refers to the side of the device intended to be in contact with the skin of the individual on which the grafts are taken, and “rear” refers to the opposite side. “Longitudinal” refers to the front-rear direction of the device and “transverse” refers to the perpendicular direction.

In reference to FIG. 1, a first design for this device is described.

According to this design, the portable cutting instrument 1 comprises a tool-rest body 2 formed from a roughly cylindrical handpiece in which is arranged, according to a central longitudinal axis X, a cylindrical tool comprising a motor 3 with a hollow axis 4. This tool is fitted to enable, in its hollow axis 4, the insertion of a cylindrical, straight, hollow metal needle 5 whose diameter ranges from 0.7 mm to 2 mm, so as to enable the circulation of grafts inside the aforementioned needle. Such a needle 5 is commonly called a “punch” in the realm of hair transplant.

Needle 5 is fitted so that it can be drawn by motor 4 in axial rotation with respect to aformentioned body 2.

Needle 5 is maintained in the hollow axis 4 by means of a link 6 arranged at the front and outside of body 2.

The front end 7 of needle 5 goes several centimeters beyond body 2. This end 7 presents a beveled and cutting circular edge 8, so as to allow for the cutting of the graft.

The device also includes a graft storage tank 9 integral with body 2, formed by a roughly cylindrical bottle. Tank 9 comprises an inlet 10 for grafts connected near the rear end 11 of needle 5 via a connector 12. In the design represented, this connector 12 is an integral part of tank 9. Nevertheless, the connector may also be a separate piece.

Tank 9 comprises a suction opening 13 connected to means of suction, these means of suction being fitted to enable the graft to directly reach tank 9. Each graft, once cut, is sucked through needle 5 and then the central axis of connector 12.

According to this design, graft inlet 10 and suction opening 13 are arranged opposite each other, thereby favouring the effiacy of the suction of the aforementioned grafts.

According to a second design, represented in FIG. 2, the tool-rest body 2 is a handpiece of the contra-angle handpiece type. Such a handpiece comprises a handle 14, a neck 15, a head 16 in which a hollow needle 17 is inserted.

The fact of using such a contra-angle handpiece, whose head 16 comprises a hollow central axis, had the advantage of giving the operator a better field of vision of the zone for the removal of each graft. In fact, the visibility is not hindered by the neck 15 and the handle 14 of the handpiece, since they form an angle with the head 16 and, in addition, the size of the head is relatively small, so that the motor driving needle 17 is located in handle 14 of the piece.

The corresponding storage tank 18 is connected near the rear end of needle 17, the aforementioned rear end roughly emerging at the centre of the graft inlet 19. Thereby, each graft, once cut, directly enters the tank 18 after having circulated by suction through needle 17. Suction opening 20 emerges here on a lateral wall 21 of tank 18.

According to a third design, represented in FIG. 3, a tank 22 is made integral with body 2 by means of a flexible cable 23 whose length does not exceed the length of body 2 since cable 23 is connected to the rear end 24 of a hollow needle 25. This arrangement is represented with a handpiece of the contra-angle handpiece type, similar to that described in relation to the second design.

This arrangement has the advantage of further improving, for the operator, the visibility of the zone of harvesting for each graft, since tank 22 is located at a distance from the head of the handpiece, and without being too far, in order to maintain the good handling of the device. In addition, it is not necessary, according to this achievement, to have a tank whose size is adjusted to that of the head, as represented in FIG. 2. It is also possible, for example, to consider a tank 22 whose size is greater than that represented in FIG. 2, able to thereby receive a larger number of grafts.

In addition, needles 17, 27 represented in FIGS. 2 and 3 are short, presenting the advantage of enabling better suction, in particular for small grafts, as well as a better concentricity resulting in a better cut.

According to a specific design, tank 9, 18, 22 on body 2 is made integral by means of impermeable connection, of a known type, in particular to avoid the penetration of air likely to harm the suction of the graft.

Moreover, needle 5, 17, 25 may be detachable with respect to body 2, facilitating its maintenance or allowing it to be changed at regular intervals.

In addition, as represented in FIG. 2, the tank may comprise an inlet 26 for a graft preservation solution, the inlet 26 being connectd to a supply pipe 27 with preservation solution, the suppy arriving from a bottle 28 containing the aforementioned solution, for example, by gravity or vacuum or by means of a peristaltic pump 29.

In particular, this preservation solution comprises isotonic solution, that conserves and moistens the grafts in the tank. It is also possible to use, in the isotonic solution, additives allowing, for example, for an improvement in the preservation.

Moreover, according to the designs represented, tank 9, 18, 22 also comprises a filtre 30 that extends over the entire transverse section of the aforementioned tank, near the suction opening. The presence of such a filtre helps keep the grafts in the tank.

The means of suction to which the suction opening of the tank 9, 18, 22 is connected may comprise a vacuum pump and means of control and measurements of the vacuum, since the connexion is made by means of a pipe 31.

In addition, the walls of the storage tank 9, 18, 22 may be transparent, enabling the operator to check the quality of the grafts as they reach the tank and check that their angle of obtention during removal is satisfactory.

1. Device to remove small hair grafts comprising a portable cutting instrument (1), the instrument comprising:
a tool-rest body (2);
a cylindrical tool with a hollow axis (4) in the body, a hollow needle (5, 17, 25) having a diameter which ranges from 0.7 mm to 2 mm, the needle being inserted in the hollow axis:
a motor (3) able to drive the needle (5, 17, 25) in rotation with respect to the body (2);
a storage tank (9, 18, 22) for grafts integral with the body (2), the tank (9, 18, 22) comprising an inlet (10, 19) for grafts connected near a rear end of the hollow needle (5, 17, 25) and a suction opening (13, 20) means for supplying graft suction being connected to the suction opening, the means of suction being fitted to enable the graft to directly reach the tank (9, 18, 22) by suction of the graft through the needle (5, 17, 25).

2. Device according to claim 1, characterised in that the tool-rest body (2) is a roughly cylindrical handpiece.

3. Device according to claim 1, characterised in that the tool-rest body (2) is a contra-angle handpiece.

4. Device according to claim 1, characterised in that the tank is integral with the body (2) by a flexible pipe (23) whose length does not exceed the length of the body (2).

5. Device according to claim 1, characterised in that the tank (9, 18, 22) is impermeably connected to the body (2).

6. Device according to claim 1, characterised in that the hollow needle (5, 17, 25) is detachable with respect to the body (2).

7. Device according to claim 1, characterised in that the storage tank (9, 18, 22) comprises a filter (30), the filter extending along the entire transverse section of the tank, near the suction opening (13, 20).

8. Device according to claim 1, characterised in that the tank (9, 18, 22) also comprises an inlet (26) for a graft preservation solution, the inlet being connected to a supply pipe (27) for preservation solution.

9. Device according to claim 8, characterised in that the preservation solution comprises isotonic solution.

10. Device according to any of claims 1 to 9, characterised in that the walls of the storage tank (9, 18, 22) are transparent.

11. Device according to claim 7, characterised in that the tank (9, 18, 22) also comprises an inlet (26) for a graft preservation solution, the inlet being connected to a supply pipe (27) for preservation solution.
